(19) United States
(12) Patent Application Publication
(10) Pub. No.: US 2007/0232941 A1
(43) Pub. Date: Oct. 4, 2007

(54) System, apparatus, and method for imaging and treating tissue

(76) Inventor: Stan Rabinovich, Edgewater, NJ (US)

Correspondence Address: EDWARDS LIFESCIENCES CORPORATION, LEGAL DEPARTMENT, ONE EDWARDS WAY, IRVINE, CA, 92614, US

(21) Appl. No.: 11/588,737

(22) Filed: Oct. 26, 2006

Related U.S. Application Data
(60) Provisional application No. 60/731,611, filed on Oct. 27, 2005.

(51) Int. Cl. A61B 5/02

(52) U.S. Cl. 600/508

(57) ABSTRACT

A catheter for treating and imaging tissue within a patient's body includes a distal portion having a tissue grasper in the form of a vacuum recess, with a close-up imaging device positioned adjacent or within the vacuum recess. The close-up imaging device provides imaging signals for imaging the tissue in front of the vacuum recess. The close-up imaging device may be in the form of an ultrasound transducer, which may be a linear or 2-dimensional array. The close-up imaging device may include imaging elements positioned around the axis of the catheter, and/or positioned on opposing sides of the tissue grasper. Another imaging device may be provided to provide “distant” images of the treatment area, with the close-up images and distant images provided to a user via one or more display screens. A tissue connector is positioned at the distal portion of the catheter.

### CROSS REFERENCE TO A RELATED PATENT APPLICATION

The present application claims priority to U.S. Provisional Patent Application No. 60/731,611, filed on Oct. 27, 2005, the disclosure of which is incorporated by reference herein in its entirety.

### FIELD OF THE INVENTION

The present invention relates to medical devices and methods. In particular, the present invention relates to a system, apparatus, and method for imaging and treating tissue, and particularly for imaging and treating adjacent tissue pieces, including heart valve leaflets and tissue around septal defects such as a patent foramen ovale (PFO).

### BACKGROUND OF THE INVENTION

Percutaneous and other minimally-invasive methods of surgery, where the surgery may be performed remotely via catheters, often include the need to fasten or otherwise treat tissue pieces which the surgeon cannot directly access. Many percutaneous and minimally-invasive medical procedures involve joining or otherwise treating tissue pieces which may move be difficult to locate and treat. For example, some tissues move with respect to the rest of a patient's body and thus can be particularly difficult to treat. For example, in some heart valve repair procedures such as edge-to-edge mitral valve repairs, adjacent heart valve leaflets are secured to each other. When such procedures are conducted on a beating heart, the heart valve leaflets move substantially with each heart beat and thus can be difficult to treat. Another example is PFO repair, where treatment may involve fastening together tissue pieces adjacent to the PFO.

Some examples of devices and methods for performing edge-to-edge mitral valve repair and similar repairs are set forth in the following patents and co-pending patent applications: U.S. Pat. No. 6,626,930, filed on May 1, 2000 and entitled, “Minimally Invasive Mitral Valve Repair Method And Apparatus”; U.S. patent application Ser. No. 10/106,583, filed on Mar. 26, 2002 and entitled “Sequential Heart Valve Leaflet Repair Device And Method Of Use”; U.S. patent application Ser. No. 10/233,879, filed on Sep. 3, 2002 and entitled “Single Catheter Mitral Valve Repair Device And Method For Use”; and U.S. patent application Ser. No. 10/389,721, filed on Mar. 14, 2003, and entitled “Mitral Valve Repair System And Method For Use.” The entire contents of the above-listed applications are expressly incorporated herein by reference. As another example, in percutaneous operations to close a patent foramen ovale (PFO), adjacent tissue pieces on either side of the PFO must be secured together via a catheter. Further description of examples of devices and methods for such PFO treatment procedures is included in co-pending U.S. patent application Ser. No. 11/174,143, filed on Jun. 30, 2005, and entitled “System, Apparatus, and Method for Repairing Septal Defects,” the entire contents of which are expressly incorporated herein by reference. The devices and methods disclosed in the current application can be incorporated into the devices, and can use the same methods of operation/treatment, as are disclosed in the above-cited patents and applications.

One difficulty in conducting tissue treatments such as percutaneous edge-to-edge mitral valve repairs is enabling the surgeon to guide and/or activate a treatment catheter to the tissue to be treated. Various visualization methods have been used with some success, including fluoroscopy and echocardiogram techniques. Such techniques are useful in positioning the catheter or other treatment device with respect to the patient's body and specific organs, such as the heart. But precise positioning with respect to smaller tissue portions, and with respect to moving tissue such as heart valve leaflets, can be difficult.

In light of the foregoing, there is presently a need for improved systems for treating and imaging tissue pieces. More specifically, there is a present need for an improved method, apparatus, and system for imaging and treating tissue. The current invention meets this need.

### BRIEF SUMMARY OF THE INVENTION

The present invention solves the problem of effectively treating and imaging tissue during catheter-based procedures within a human body, and particularly tissue which is relatively small and/or moving with respect to the body. Additionally, the present invention provides a device capable of imaging and treating tissue via a catheter from a remote insertion location.

In one aspect, the present invention is directed to a system for imaging and treating tissue and includes a combined imaging and treatment catheter having a tissue treatment apparatus and a tissue close-up imaging apparatus. The close-up tissue imaging apparatus can include an ultrasound transducer positioned on or adjacent the tissue treatment apparatus. The system can further include a second imaging apparatus which provides a broader view of the organ or other body structure on or in which the tissue being treated is located.

In one aspect, the present invention is directed to a system for imaging and repairing tissue and includes a combined imaging and treatment catheter having at least one tissue grasper in the form of a vacuum recess for stabilizing tissue, with an imaging device positioned adjacent and/or within the vacuum recess.

In another aspect, the present invention discloses a catheter for treating tissue within the heart of a patient and includes an elongated body having a distal end, at least one imaging device at the distal end, at least one suction recess formed on the distal end, at least one needle port located proximate to the suction recess, at least one needle lumen having at least one needle positioned therein in communication with the needle port, at least one needle receiving port having at least one needle catch located therein positioned proximate to the suction recess, and at least one actuator member in communication with the needle.

Other objects, features, and advantages of the present invention will become apparent from a consideration of the following detailed description.

### BRIEF DESCRIPTION OF THE DRAWINGS

FIG. 1 illustrates an imaging and treatment catheter advanced within a patient's vasculature and into a patient's heart according to an embodiment of the invention;

FIG. 2 is a close-up perspective view, in partial cross section, of the distal portion of the imaging and treatment catheter of FIG. 1 advanced within a patient's vasculature.

FIG. 3 is a side view, in partial cross section, of an imaging treatment catheter according to an embodiment of the invention;

FIG. 4 illustrates a system for treating and imaging tissue according to an embodiment of the invention;

FIG. 5 is a side view of a distal end of an imaging and treatment catheter according to an embodiment of the invention;

FIG. 6 is a side view of a distal end of an imaging and treatment catheter according to an embodiment of the invention;

FIG. 7 is a side view of a distal end of an imaging and treatment catheter according to an embodiment of the invention;

FIG. 8a is a side view of a distal end of an imaging and treatment catheter according to an embodiment of the invention;

FIG. 8b is a front view, in partial cross-section, of a distal end of the imaging and treatment catheter depicted in FIG. 8a;

FIG. 9 is a side view of a distal end of an imaging and treatment catheter according to an embodiment of the invention; and

FIG. 10 is a perspective view of a distal end of an imaging and treatment catheter according to an embodiment of the invention.

### DETAILED DESCRIPTION OF THE INVENTION

The invention is an apparatus, system, and method for imaging and treating selected tissue. More specifically, the invention provides for percutaneous or other minimally-invasive imaging and treatment of tissue within a patient's body.

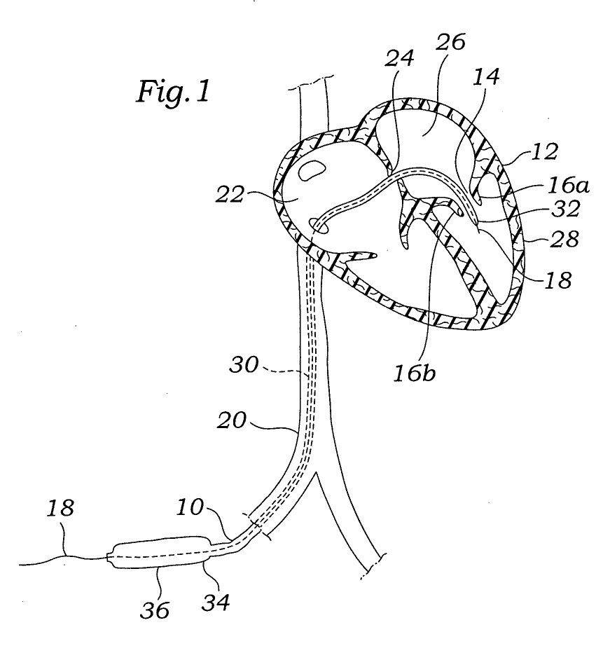

FIG. 1 depicts a catheter 10 according to the invention being advanced through a patient's vasculature to a heart 12 and into a mitral valve annulus 14 and adjacent mitral valve leaflets 16a, 16b. A guidewire 18 has previously been advanced through the vasculature by passing up the inferior vena cava 20, through the right atrium 22, through the septum 24 (via a natural opening such as a PFO or via a perforation created by the physician), through the left atrium 26, and into the mitral valve annulus 14. Note that other introductory routes, including other percutaneous and minimally invasive routes, are also within the scope of the invention. Depending on the particular embodiment, the device may also be introduced through the heart wall 28, as may be the case in a minimally-invasive surgical procedure conducted through a patient's chest cavity. The particular route selected for introduction of the device depends on various factors, including the type and location of the tissue to be treated as well as the condition of the patient.

The treatment catheter 10 comprises a generally elongated body 30 having a distal end 32 and a proximal end 34. A handle 36 is located at the proximal end 34. The treatment catheter 10 has sufficient length to reach the mitral valve annulus 14 from outside the patient's body via the particular route selected. For a percutaneous route, the treatment catheter will generally have a length on the order of 60 to 120 cm, depending on the patient, the valve to be treated, the access route selected, and other factors such as user preference, etc. Treatment catheter lengths of 90 to 100 cm, and even 90 to 120 cm, may be used depending on the particular application. Other access routes may require different lengths. The elongated body 30 and distal end 32 have a diameter that is small enough to pass through the particular blood vessels and/or openings of the particular access route selected. While percutaneous approaches through the inferior vena cava, as depicted in FIG. 1, can accommodate diameters of 12 to 24 Fr, other approaches may accommodate and/or require smaller or larger diameters.

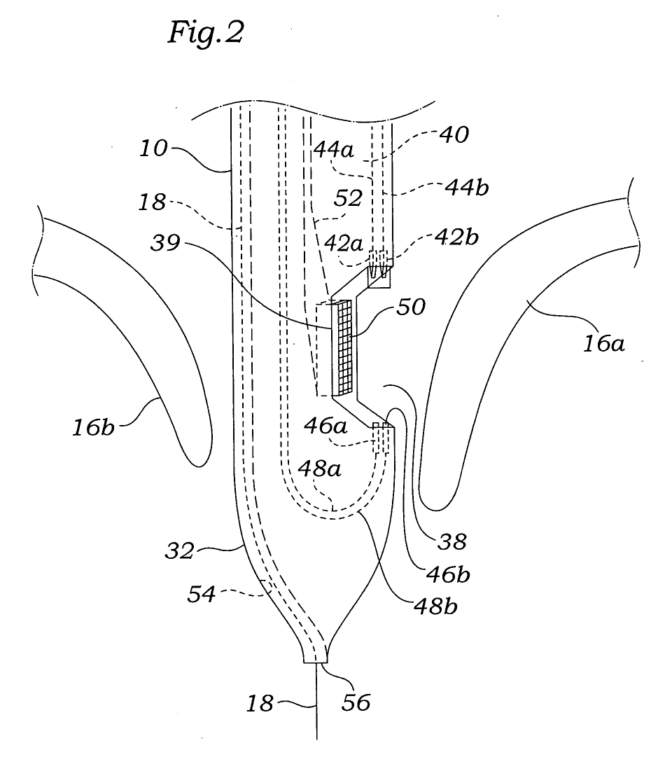

FIG. 2 depicts a close-up view of the distal end 32 of the treatment catheter 10 of FIG. 1, with the distal end 32 passing between the mitral valve leaflets 16a, 16b. The distal end 32 includes a tissue stabilizing and/or capturing apparatus in the form of a tissue grasper, which in the particular embodiment depicted includes a vacuum recess 38 which leads to a vacuum lumen 40. When vacuum is applied to the vacuum recess 38 via the vacuum lumen 40, tissue such as the heart valve leaflet 16a can be drawn into and releasably held within the vacuum recess 38. Once captured, the tissue will generally be held within vacuum recess 38 until the application of vacuum is discontinued.

The catheter distal end 32 also includes a tissue treating apparatus including one or more needles 42a, 42b secured to one or more corresponding needle drivers 44a, 44b which can longitudinally advance and retract the needles 42a, 42b across the vacuum recess 38. Opposite of the needles 42a, 42b across the vacuum recess 38 are one or more needle catchers 46a, 46b, each of which is configured to be speared by its respective needle 42a, 42b and drawn back across the vacuum recess 38 when the needles 42a, 42b are retracted. The needle catchers 46a, 46b are secured to suture ends 48a, 48b. In the particular embodiment depicted, the suture ends 48a, 48b run longitudinally through the catheter body 30. The suture ends 48a, 48b may be opposite ends of a common suture line, or they may be portions of different suture lines.

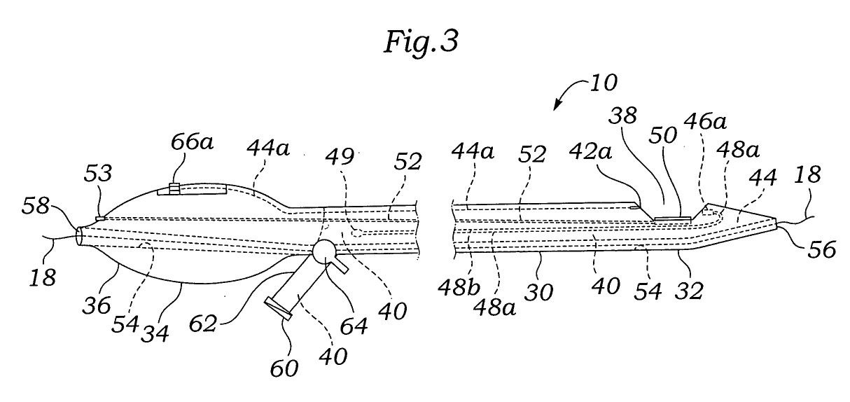

An imaging device in the form of an ultrasound transducer 50 is positioned within the vacuum recess 38. The ultrasound transducer 50 is connected to a carrier 52, such as a wire cable or fiber-optic cable, through which signals can go to and/or from the ultrasound transducer 50. In the embodiment of FIG. 2, the ultrasound transducer 50 transmits ultrasonic signals out of, and receives returning signals back through, the vacuum recess 38. The ultrasound transducer 50 can thus image tissue such as the heart valve leaflet 16a that is adjacent the vacuum recess 38. In the particular embodiment of FIG. 2, the top (imaging) surface of the ultrasonic transducer 50 is generally level with the lower edge 39 of the vacuum recess 38. However, depending on the particular embodiment, the transducer 50 could be positioned at other locations within the vacuum recess 38, including being entirely below lower edge of the vacuum recess opening, or being positioned with the imaging surface above the lower edge of the vacuum recess opening (as is depicted in FIG. 3).

In the embodiment described in FIGS. 1 and 2, the catheter is depicted and described in use to treat a mitral valve. The invention is not limited to mitral valve treatments, however, and is applicable to treatment of other tissue within a patient's body such as tissue from other heart valves, tissue adjacent a patent foramen ovale (PFO), etc.

The embodiment of FIGS. 1 and 2 includes a guidewire 18, with a guidewire lumen 54 passing through the catheter body 30 and terminating in a distal guidewire opening 56 at the catheter distal end 32. Note that the guidewire 18 does not have to be present in all embodiments, such as where the treatment catheter 10 is steerable on its own to the tissue to be treated.

FIG. 3 depicts another view of the treatment catheter 10, including the generally elongated body 30, distal end 32, and a proximal end 34. The treatment catheter 10 has sufficient length to reach the mitral valve leaflets or other tissue to be treated, from outside the patient's body via the particular route selected. For a percutaneous route, the treatment catheter will generally have a length on the order of 60 to 120 cm, with lengths of 90 to 100 cm or 90 to 120 cm being typical. Other access routes may require different lengths. The elongated body 30 and distal end 32 have a diameter that is small enough to pass through the particular blood vessels and/or openings of the particular access route selected. While percutaneous approaches through the inferior vena cava, as depicted in FIG. 1, can accommodate diameters of 12 to 24 Fr, other approaches may accommodate and/or require smaller or larger diameters. The vacuum lumen 40 passes through the catheter body 30 from the distal end 32 to the proximal end 34, where it terminates in a vacuum attachment adaptor 60 positioned on a y-connector 62. The guidewire lumen 54 is depicted passing through the catheter body 30 between the proximal guidewire opening 58 at the proximal end 30 and the distal guidewire opening 56 at the distal end 32.

The handle 36 may include a valve 64 and/or other device (such as a pump, etc.) to provide and/or control the application of vacuum to the vacuum lumen 40. For systems having multiple vacuum recesses where independent control of the application of vacuum thereto is desired (such as that disclosed in U.S. Pat. No. 6,626,930 entitled “Minimally Invasive Mitral Valve Repair Method And Apparatus,” the contents of which are hereby incorporated in their entirety), the valve and/or other vacuum provider/control devices may be configured to provide such independent control and application. For example, a single valve could have multiple positions to independently control vacuum to multiple vacuum recesses. Multiple valves might also be used for such independent control.

The handle 36 depicted in FIG. 3 includes a carrier connector 53 where the carrier line 52 can be connected to an outside carrier line. The handle 36 also includes a knob 66a which, when advanced distally or retracted proximally, causes, via the needle driver 44a, the needle 42a to be advanced or retracted. A corresponding knob 66b can be included to provide independent control of advancement and retraction of needle driver 44b and needle 42b. (Note that elements 42b, 44b, and 66b are not visible in the side view of FIG. 3.) In the embodiment depicted in FIG. 3, the suture ends 48a, 48b are opposite ends of a single suture line which forms a loop 49 near the proximal portion 36 of the catheter 10.

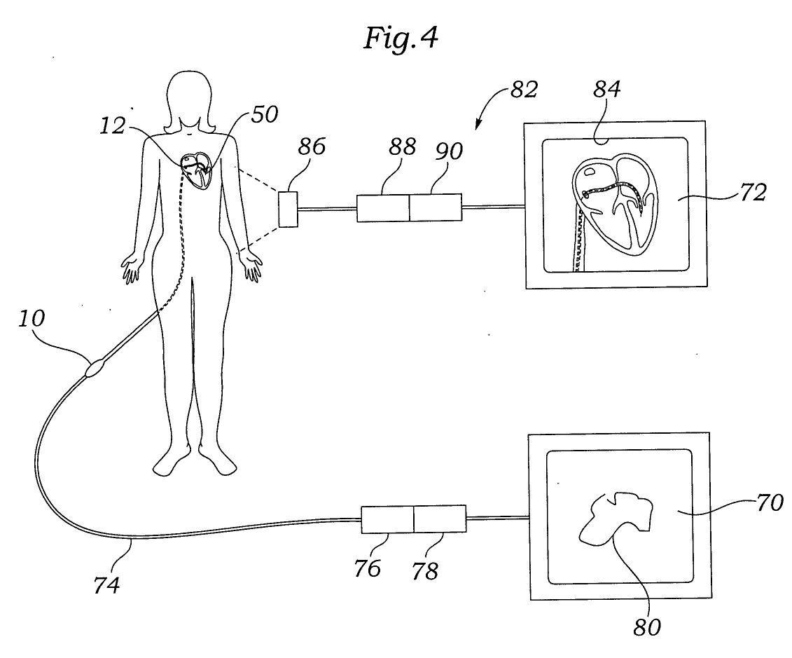

FIG. 4 depicts a system according to the invention. The system includes the treatment catheter 10 as well as a first image display 70 and a second image display 72. The treatment catheter 10 is connected to the multiple image displays via a carrier connector line 74. An ultrasound transducer controller 76, such as a microprocessor or other control system, transmits signals through the carrier connector line 74 to create and control the signals transmitted from the ultrasound transducer 50. A transducer return signal processor 78 (which can be an integral part of the ultrasound transducer controller) receives returning signals through the carrier line corresponding to the transducer returns and translates the signals into a “close-up” tissue image 80 which is depicted on the first image display 72. The close-up tissue image 80 corresponds to the tissue directly in front of the ultrasound transducer 50, so that where the ultrasound transducer 50 is located on the catheter 10 within or adjacent the catheter vacuum recess, the tissue adjacent the catheter vacuum recess is imaged. For example, where the catheter distal end 32 is positioned as depicted in the embodiment depicted in FIG. 2, the close-up tissue image 80 provided to the first image display 70 would correspond to the valve leaflet 16a adjacent the vacuum recess 38.

The system of FIG. 4 further includes a second imaging apparatus 82 that is directed to creating an image of the catheter and the organ or other body feature in which the specific tissue being treated is located. The second imaging apparatus 82 provides a second image 84 to the second image display 72, with the second image 84 corresponding to the position of the catheter within the body and/or body organ. Thus, the first image 80 on the first image display 70 is a close-up view of the tissue being treated, while the second image 84 on the second image display 72 is a broader view showing more of the surrounding features, including the organ (which in the embodiment depicted is a heart 12), the catheter 10, etc.

A user can thus use the second image display 72 to determine if the catheter is properly positioned with respect to the organ and tissue to be treated. The user can then use the first image display 70 to “fine-tune” the position of the catheter with respect to the particular tissue to be treated. The user can also, or alternatively, use the first image display 70 to track tissue movements, which may be particularly helpful in treating tissue structures such as heart valve leaflets which tend to move with respect to other body features.

In the embodiment depicted in FIG. 4, the second image is provided by an ultrasound imaging system having an ultrasound transducer 86, an ultrasound transducer controller 88, and a transducer return signal processor 90. However, it is not necessary to the invention that the first and second images are provided by the same type of imaging systems. For example, the first imaging system could be an ultrasound imaging system, but the second imaging system could be rely on totally different imaging technology such as x-ray, fluoroscopy, etc. Using different imaging technologies for the two imaging system may reduce the chances of interference that might occur if similar imaging technologies (such as ultrasound imaging systems using similar frequencies) were employed for both systems.

Note that any of the carrier lines and/or similar connectors could be eliminated and replaced with wireless communication links, such as where the ultrasound transducer 50, ultrasound transducer controller 76, and/or return signal processor 78 were linked via wireless communications such as Bluetooth.

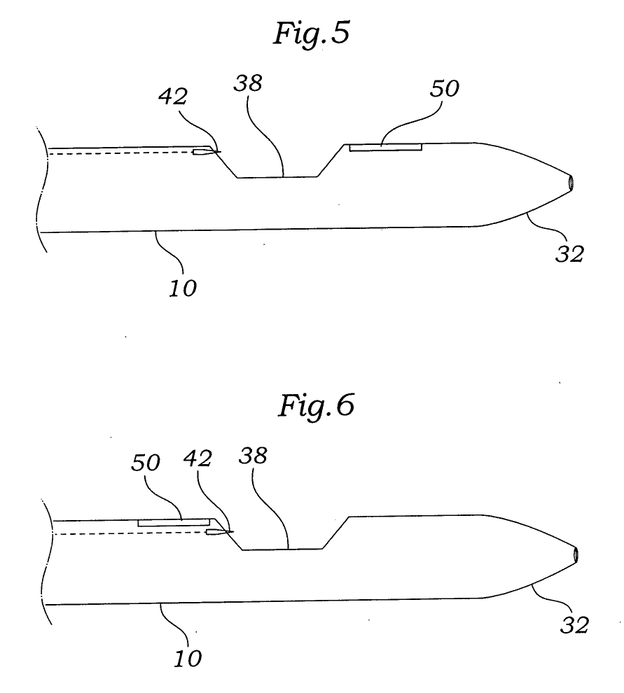

In the embodiment depicted in FIGS. 1 and 2, the ultrasound transducer 50 was positioned within the vacuum recess 38, which in the particular catheter 10 of FIGS. 1 and 2 placed the ultrasound transducer 50 adjacent the tissue treatment apparatus (i.e., suturing needle 42) and provided a close view of the heart valve leaflet 16a to be grasped and treated. Positioning the ultrasound transducer 50 within the vacuum recess 38 may also have the added benefit of allowing the user to clean the transducer surface by applying vacuum to the vacuum lumen 40, or providing a fluid flow from the vacuum lumen 40 to the vacuum recess 38, so that fluid flowing either in or out of the vacuum recess 38 will sweep potentially view-blocking material clear of the ultrasound transducer 50. The transducer 50 is also positioned with sufficient spacing in the vacuum recess all around so that vacuum can be applied, and fluid can flow, around the edges of the transducer 50. Other locations of the ultrasound transducer may also be acceptable, depending on the particular application. For example, an ultrasound transducer could be located on the catheter distal end just distal of or proximal of the tissue stabilizer and/or treatment apparatus. Such examples are depicted in FIG. 5, where an ultrasound transducer 50 is positioned just distally from the vacuum recess 38 and treatment needles 42, and in FIG. 6, where an ultrasound transducer 50 is positioned just proximally from the vacuum recess 38 and treatment needles 42. In a further embodiment (not depicted) a single catheter could have multiple transducers at multiple locations. For example, a single catheter could have multiple transducers mounted around and/or at opposing sides of the vacuum recess, e.g., a first transducer mounted distal of the vacuum recess, with a second transducer mounted proximal of the vacuum recess.

The system could be used in a variety of procedures. For example, the system could be used to secure adjacent heart valve leaflets together. The catheter could be advanced percutaneously through the patient's vascular system and into the patient's heart, with the cardiologist or other user monitoring the catheters position in the patient's body via second display. The user positions the catheter distal end within the valve annulus so that the treatment portion is adjacent the valve leaflets. This position can be determined with the second display. The user can use the catheter-mounted ultrasound transducer and associated first display to make final adjustments to (“fine-tune”) and/or confirm the catheter's distal end position in order to position the catheter treatment element at a desired location adjacent the heart valve leaflet or leaflets to be treated. The movements of a first heart valve leaflet are monitored via the catheter-mounted ultrasound transducer and first display. When the user determines via the first display that the catheter and leaflet are in proper position with respect to each other, the user activates the grasping element of the catheter in order to grasp the valve leaflet. The user can then use the first display and/or second display to confirm that the leaflet has been correctly grasped, including determining whether the grasp is secure and whether the leaflet has been grasped at a desired location on the leaflet, which is typically in the middle portion of the leaflet edge. If the grasp is not secure and/or properly positioned, the user can release the leaflet, reconfirm and/or fine-tune the catheter position via the first display, monitor the heart valve leaflet position with respect to the catheter via the first display, and then reactivate the grasping apparatus to re-grasp the leaflet. Once a proper grasp has been confirmed, the user can activate deployment of the securing element or elements, such as the deployment of the needle and suture used in the device from FIGS. 1 and 2. Depending on the particular securing element or elements used, the user can use the first display and/or second display to confirm deployment of the securing element or elements, and to assess and/or confirm the quality (including location, etc.) of the attachment of the securing element to the leaflet. For the embodiment of FIGS. 1 and 2, the user might follow the above procedure for a first leaflet, then reposition the catheter distal end to place it adjacent a second leaflet, and then repeat the above process for the second leaflet.

For a catheter configured to simultaneously hold two leaflets for treatment, such as several of the catheters depicted and described in U.S. Pat. No. 6,626,930 (the entire contents of which are incorporated herein by reference), the process could be modified so that both leaflets are grasped, either simultaneously or sequentially, with the first display and/or second display used to confirm the nature and quality of the grasping. The user can then deploy the attachment elements, simultaneously or sequentially, with the first display and/or second display used to confirm the nature and quality of the deployment of the attachment elements on or into the leaflet tissue.

The above-discussed procedures involve treating heart valve leaflets. However, other procedures are also within the scope of the invention, such as treating PFOs or other heart procedures, or treatments of other body tissue. The procedural steps would generally be similar, using the following steps or subcombinations thereof. The user advances the catheter to a desired position adjacent the tissue to be treated, determines the general catheter location via the second display, fine-tunes and/or confirms the catheter distal end position using the first display, monitors and/or confirms the tissue location with respect to the catheter distal end via the first display, grasps the desired tissue, assess and/or confirms the grasping of the desired tissue via the fist display, deploys a desired tissue treatment element onto or into the targeted tissue, and assesses and/or confirms the tissue treatment deployment onto or into the targeted tissue via the first display. The steps could be performed in the above order, or the order could be varied according to the desires of the user and the particular procedure involved.

The invention could be sold as a kit, with the kit including the combined imaging/treatment catheter with instructions on how to use the catheter in combination with available displays at a hospital, as well as instructions detailing the procedural steps involved in performing leaflet repair and/or other tissue treatment procedures such as those discussed above.

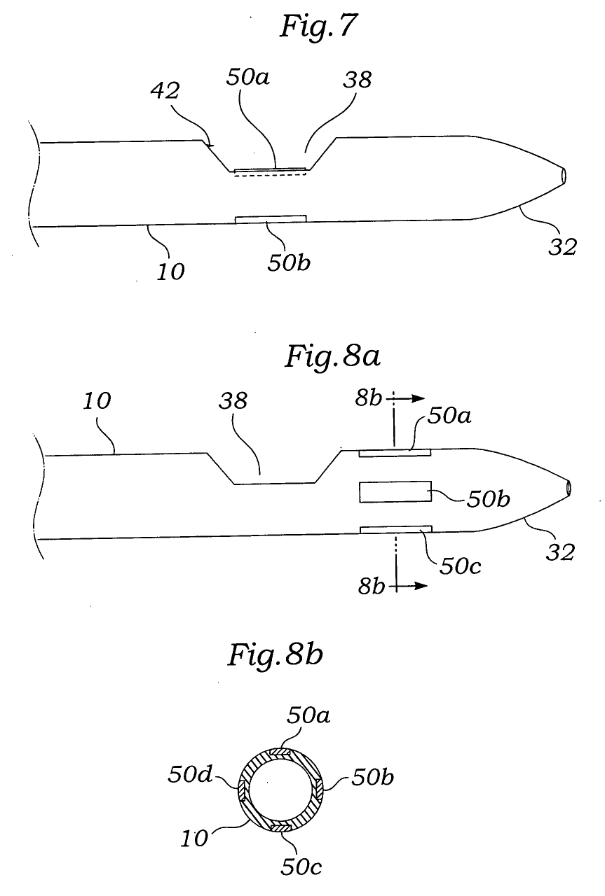

Multiple transducers could be positioned at various locations on the catheter. For example, two transducers 50a, 50b (or two groups thereof) could be positioned on opposing sides of the catheter as in FIG. 7, or multiple transducers 50a, 50b, 50c, 50d could be positioned about the circumferential radius of the catheter as in FIGS. 8a and 8b, in order to fine-tune catheter position within a structure, such as where a user may want to position the catheter directly in the center of a structure such as a valve annulus.

Various types of imaging systems could be used in conjunction with the invention, depending on the particular application. For example, an ultrasound transducer could be used to provide images from the catheter itself, as depicted in FIGS. 1 and 2. The specific type of ultrasound transducer selected could vary depending on the requirements of the particular application. For example, there may be size restrictions presented. An ultrasound transducer and its associated connectors (carrier line, etc.) will have to be relatively small in order to fit on or in a catheter intended for percutaneous use.

In a relatively simple approach, a single-element transducer could be used for applications where high resolution may not be required for the first image (i.e., close-up) display. For example, in an edge-to-edge leaflet repair procedure where the goal is to grasp a heart valve leaflet as it flaps in response to blood flow, the user may not need to visually confirm very many details of the shape of the leaflet being targeted. If the user knows from the second imaging system that the catheter is adjacent the heart valve annulus, an indication from the first imaging system that some sort of structure is alternatively appearing and disappearing from the area in front of the catheter vacuum recess may be sufficient for the surgeon or other user to conclude that the catheter is in proper position to grasp the targeted leaflet. If the disappearing/reappearing structure is appearing and disappearing in time to the beating of the patient's heart, the physician or other user may reasonably conclude that the structure is the heart valve leaflet. Accordingly, high resolution imaging of the targeted tissue structure may not be necessary in all applications, so that even a single-element transducer may be sufficient to provide the imaging for the first image display.

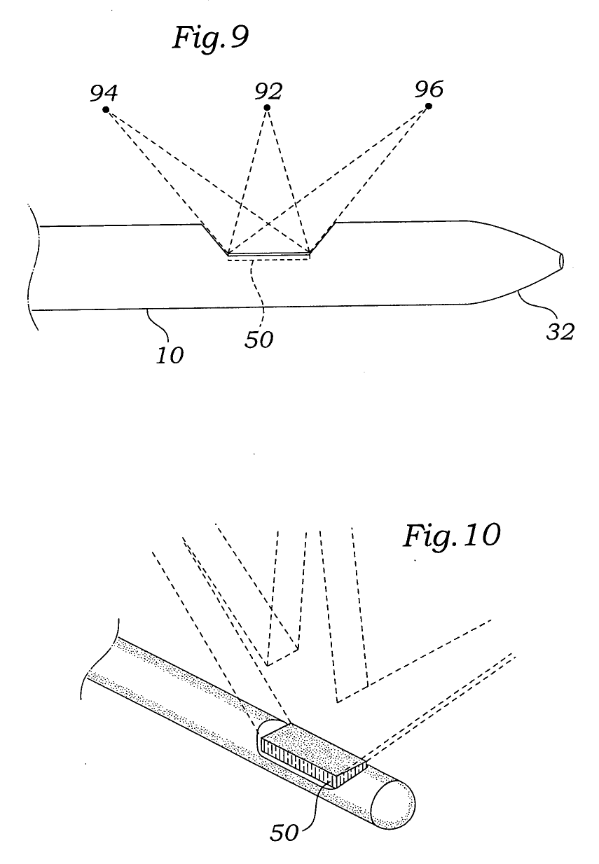

Another option for ultrasound transducer is a linear transducer array, which has multiple elements (such as multiple piezo-electric crystals) arranged in line, such as a flat or curved line. The elements are fired in precise sequences, and each one then “listens” for an echo. A digital scan converter processes the returning electrical signals, and produces an image on the display monitor. An ultrasound imaging system repeats the entire send/receive cycle for the linear array of elements many times each second, and updates the image on the screen continuously. A linear array will typically have better resolution than a single-element transducer. Linear transducers can also be aimed to some extent along the direction of the transducer line without requiring movement of the transducer, so that the user can adjust the view to provide an image directed toward the area directly next to 92, proximal of 94, and/or distal of 96 the transducers location, as depicted FIG. 9.

Two-dimensional transducer arrays may also be used. Two-dimensional transducer arrays generally have increasing aiming and resolution abilities over corresponding linear and/or single element transducers. A two-dimensional array manufactured by Tetrad Corporation is depicted in FIG. 10 and has the following characteristics:

TABLE A

| | |
|---|---|
| Center Frequency (−6 dB) | 6.5 MHz +/− 0.5 MHz |
| Bandwidth | >50% |
| Number of Elements | 64 |
| Pitch | 0.11 mm |
| Elevation Width | 2.5 mm |
| Elevation Focus | Flat Face |
| Array Package Size | 2.5 mm wide, 8.1 mm long, 1.6 mm deep |
| Transducer Surface Size | 2.5 mm wide × 8.1 mm long |

Such a device can provide detailed imaging of structures within the body, such as chambers within a human heart. But the device dimensions may be too large for many percutaneous applications. In addition to the array itself, the supporting hardware (including the carrier line assembly, which could have sixty-four (64) separate conductor lines if each array element was served by an individual conductor line) might further drive up the size of the assembly. By going with a smaller array, an imaging device could be sized to fit on or in a small catheter while still providing sufficient resolution for close-up imaging. An example of one such smaller device might have the following characteristics:

TABLE B

| | |
|---|---|
| Center Frequency (−6 dB) | 10 MHz +/− 0.5 MHz |
| Bandwidth | >50% |
| Number of Elements | 32 |
| Pitch | 0.075 mm |
| Elevation Width | 1.0 mm |
| Elevation Focus | Flat Face |
| Array Package Size | 1.3 mm wide, 2.6 mm long, 1.4 mm deep |
| Transducer Surface Size | 1.3 mm wide × 2.6 mm long |

A device with the characteristics set forth in Table B could make reasonable quality images to a range of 25 mm from the surface of the transducer. It could sweep out a 90 degree sector, and could make real-time images of body structures such as moving valve leaflets.

While the invention has been described with reference to particular embodiments, it will be understood that various changes and additional variations may be made and equivalents may be substituted for elements thereof without departing from the scope of the invention or the inventive concept thereof. For example, while the invention is specifically discussed in application with repair of heart valve leaflets and septal defects such as PFOs, it has applicability in other areas where it is desired to repair tissue. In addition, many modifications may be made to adapt a particular situation or device to the teachings of the invention without departing from the essential scope thereof. Therefore, it is intended that the invention not be limited to the particular embodiments disclosed herein, but that the invention will include all embodiments falling within the scope of the appended claims.

What is claimed is:

1. A method of treating tissue, the method comprising:
providing a catheter device having a proximal end, a distal end, at least one vacuum recess near the distal end, a first imaging apparatus near the distal end, the first imaging apparatus positioned within or adjacent the vacuum recess, and a connector near the distal end;
advancing the catheter through a patient's circulatory system to position the distal end and first imaging apparatus adjacent the tissue to be treated;
collecting a first set of imaging data, wherein the first set of imaging data is collected from the tissue to be treated, and the first set of imaging data is collected via the first imaging apparatus;
providing a first image of the tissue to be treated using the first set of imaging data;
applying a vacuum to the first vacuum recess to stabilize a first tissue portion; and
securing the connector to the first tissue portion.

2. The method of claim 1, wherein the tissue to be treated is on or in a body organ, and the method further comprises:
providing a second imaging apparatus, the second imaging apparatus positioned away from the catheter device; and
collecting a second set of imaging data, wherein the second set of imaging data is collected from the body organ, and the second set of imaging data is collected via the second imaging apparatus.

3. The method of claim 2, further comprising:
providing a second image of the tissue to be treated using the second set of imaging data.

4. The method of claim 3, wherein the first image of the tissue to be treated is presented on a first display, and the second image of the tissue to be treated is presented on a second display.

5. The method of claim 3, wherein the first image and the second image of the tissue to be treated are both presented on a first display.

6. The method of claim 1, whereby the first imaging apparatus is positioned within the vacuum recess, the connector comprises suture attached to a needle, and the needle is configured to pass across at least a portion of the vacuum recess and in front of the first imaging apparatus,
and wherein the step of securing the connector to the first tissue portion comprises passing the needle across the portion of the vacuum recess and in front of the first imaging apparatus, whereby the needle can be imaged by the first imaging apparatus.

7. A device for treating tissue, the device comprising:
an elongated body having a proximal end and a distal end;
a tissue grasper positioned near the distal end of the elongated body; and
an imaging apparatus positioned within or adjacent the first tissue grasper.

8. The device of claim 7, wherein the tissue grasper comprises a vacuum recess.

9. The device of claim 8, wherein the imaging apparatus comprises a first imaging element positioned within the vacuum recess.

10. The device of claim 9, wherein the device further comprises:
a first needle configured to penetrate tissue held by the tissue grasper, wherein the first needle is configured to be passed across at least a first portion of the vacuum recess and in front of the first imaging element, whereby the first needle can be viewed by the first imaging element as the first needle is passed across the first portion of the vacuum recess.

11. The device of claim 9, wherein the first imaging element is positioned on a first side of the catheter, the imaging apparatus further comprises a second imaging element positioned on a second side of the catheter, and the first side of the catheter is an opposite side to the second side of the catheter.

12. The device of claim 8, wherein the imaging apparatus comprises multiple imaging elements positioned about a circumferential radius of the catheter.

13. The device of claim 7, wherein the imaging apparatus comprises an ultrasound transducer.

14. The device of claim 13, wherein the ultrasound transducer is a linear array.

15. The device of claim 13, wherein the ultrasound transducer comprises an array having at least two dimensions.

16. The device of claim 9, wherein the imaging apparatus comprises an ultrasound transducer, and the ultrasound transducer comprises a first transducer element and a second transducer element, wherein the first transducer element is positioned on the catheter just distal of the vacuum recess, and the second transducer element is positioned on the catheter just proximal of the vacuum recess.

17. A system for treating tissue, the system comprising:
a catheter configured to be advanced within a patient's body, the catheter comprising a proximal end and a distal end, and further comprising a tissue grasping element at the catheter distal end, and having a first imaging apparatus at or adjacent the tissue grasping element, the first imaging apparatus configured to provide first imaging signals; and
a first display configured to receive the first imaging signals and to display a first image derived from the first imaging signals.

18. The system of claim 17, further comprising:
a second imaging apparatus, wherein the second imaging apparatus is positioned away from the catheter, the second imaging element configured to provide second imaging signals.

19. The system of claim 18, further comprising a second display configured to receive the second imaging signals and to display a second image derived from the second imaging signals.

20. The system of claim 18, wherein the first display is configured to receive the second imaging signals and to display a second image derived from the second imaging signals.
